(19) United States
(12) Patent Application Publication
(10) Pub. No.: US 2015/0164430 A1
(43) Pub. Date: Jun. 18, 2015

(54) METHOD FOR CLASSIFYING USER MOTION

(71) Applicant: Lark Technologies, Inc., Mountain View, CA (US)

(72) Inventors: Julia Hu, Mountain View, CA (US); Jeff Zira, Mountain View, CA (US); Alvin Lacson, Mountain View, CA (US)

(21) Appl. No.: 14/315,195

(22) Filed: Jun. 25, 2014

Related U.S. Application Data
(60) Provisional application No. 61/916,707, filed on Dec. 16, 2013.
(60) Provisional application No. 61/839,155, filed on Jun. 25, 2013.

(51) Int. Cl. A61B 5/00; A61B 5/11

(52) U.S. Cl. CPC A61B 5/7264; A61B 5/1123; A61B 5/7278; A61B 5/7282; A61B 5/1126; A61B 5/0004; A61B 5/6898; A61B 5/6801; A61B 5/7221; A61B 5/7275; A61B 5/7246

(57) ABSTRACT

A method for classifying motion of a user includes: during a second time interval, receiving a set of sensor signals from a set of motion sensors arranged within a wearable device; from the set of sensor signals, generating a set of quaternions corresponding to instances within the second time interval; generating a set of motion features from the set of quaternions; transforming the set of motion features into a second action performed by the user within the second time interval; and transmitting a flag for the second action to an external computing device in response to a difference between the second action and a first action, the first action determined from data received from the set of motion sensors during a first time interval immediately preceding the second time interval.

### CROSS REFERENCE TO RELATED APPLICATIONS

This Application claims the benefit of U.S. Provisional Application No. 61/916,707, filed on 16 Dec. 2013, and to U.S. Provisional Application No. 61/839,155, filed on 25 Jun. 2013, both of which are incorporated in their entireties by this reference.

### TECHNICAL FIELD

This invention relates generally to the field of digital health, and more specifically to a new and useful method for classifying user motion in the field of digital health.

### BRIEF DESCRIPTION OF THE FIGURES

FIG. 1 is a flowchart representation of a first method of the invention; and

FIG. 2 is a flowchart representation of a second method of the invention.

FIG. 3 is a flowchart representation of a third method of the invention.

FIGS. 4A and 4B are flowchart representations of variations of the third method.

FIG. 5 is a flowchart representation of one variation of the third method.

### DESCRIPTION OF THE EMBODIMENTS

The following description of the embodiments of the invention is not intended to limit the invention to these embodiments, but rather to enable any person skilled in the art to make and use this invention.

### 1. First Method

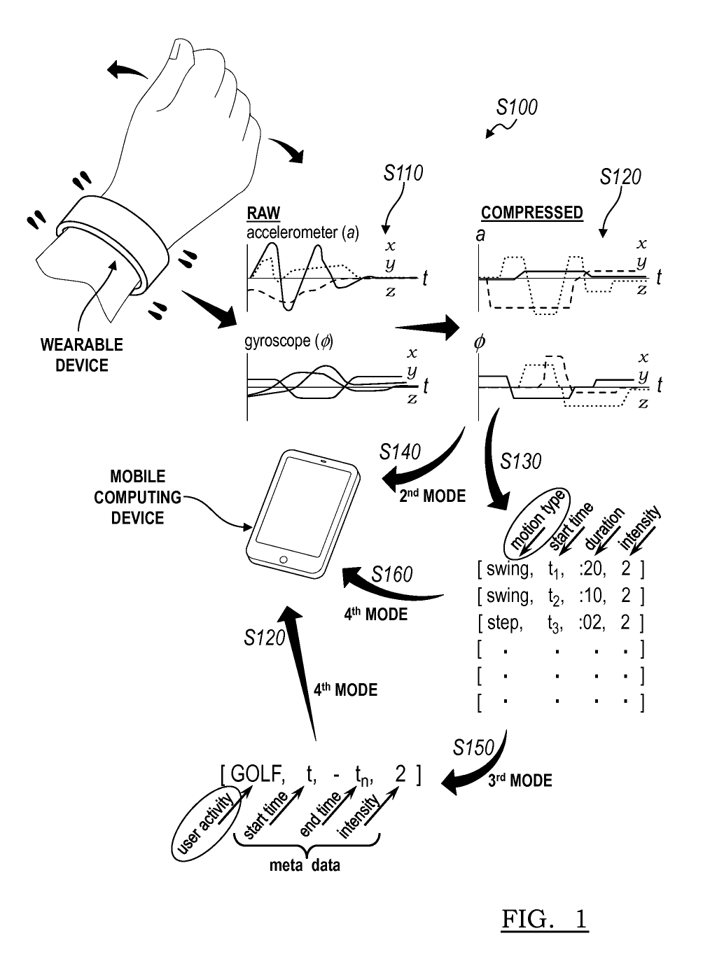

As shown in FIG. 1, a first method S100 for classifying a user action includes: recording a set of raw motion data through a sensor incorporated into a wearable device in Block S110; generating compressed motion data from the set of raw motion data in Block S120; in a first mode, correlating the compressed motion data with a motion type in Block S130; in a second mode, transmitting the compressed motion data to a paired mobile computing device if the compressed motion data is not correlated with a motion type in Block S140; in a third mode, correlating the motion type with a user activity in Block S150; in a fourth mode, transmitting the motion type to the paired mobile computing device if the motion type is not correlated with a user activity in Block S160; in a fifth mode, transmitting the user activity to the paired mobile computing device in Block S170.

Generally, the first method S100 enables a wearable device to compress raw motion data at various levels prior to transmission to a mobile computing device paired to the wearable device. For example, the first method S100 can handle transmission of raw motion data, compressed motion data (e.g., quaternions), extrapolated motion types, and/or extrapolated actions (e.g., including an identified action, a start time, an end time, a duration, and/or an intensity, etc.). The first method S100 can execute on a wearable device incorporating a processor, a wireless communication module, a battery, and one or more motion sensors, such as an accelerometer and/or a gyroscope. The wearable device can additionally or alternatively include a magnetometer configured to measure presence of a magnetic field, such as to determine orientation, a barometer configured to measure elevation, and/or any other suitable sensor. The wearable device can be a wrist-type wearable device, such as described in U.S. Provisional Application No. 61/710,867, filed on 8 Oct. 2012, which is incorporated in its entirety by this reference, or any other suitable head-, arm-, foot-, shoe-, torso-, or other wearable device. The wearable device can thus record user motion through one or more motion sensors and store user motion data locally prior to compression (via the first method S100) and transmission to the mobile computing device.

The first method S100 can also handle data communications with a smartphone, a tablet, or any other suitable mobile computing device, such as described in U.S. patent application Ser. No. 13/100,104, filed on 15 Jul. 2011, in U.S. patent application Ser. No. 14/048,956, filed on 8 Oct. 2013, and in U.S. patent application Ser. No. 14/289,384, filed on 28 May 2014, which are incorporated in their entireties by this reference. For example, the first method S100 can transmit data of various compression levels over Bluetooth, Wi-Fi, or any other suitable wireless communication protocol to the mobile computing device that is paired or otherwise associated with the wearable device.

The first method S100 can dynamically adjust compression levels of local user motion data to limit an amount of data transmitted from the wearable device to the mobile computing device without substantially sacrificing accuracy of motion type or activity identification. Because wireless transmission can be power-intensive, minimizing an amount of data transmitted from a wearable device can serve to extend the battery life of the device, which can be particularly important for a wearable device meant to be worn for extended periods of time and/or of a minimal size. Therefore, by dynamically processing raw motion data locally on the wearable device, such as into compressed motion data, a motion type, or an activity based on a degree of confidence in extrapolated data prior to transmitting data to a paired mobile computing device, the first method S100 can reduce the amount of transmitted data and thus extend the battery life of the wearable device. In one implementation, Block S120 implements a lossy or lossless compression algorithm to reduce the size of recorded user motion data, Block S130 implements a motion type recognition algorithm to characterize a set of raw or compression motion data into a motion type (e.g., a walking motion, a running motion, a swinging motion, a drinking motion, a typing motion, etc.), and Block S150 implements an activity recognition algorithm to classify raw, compressed, or motion type data into a user activity (e.g., hiking, biking, eating, playing tennis, working at a computer, etc.). The first method S100 can thus selectively apply these algorithms locally to user motion data stored on the wearable device—based on a determined degree of confidence in extrapolated data—to reduce the size (i.e., length, bits) of user motion data transmitted to the mobile computing device. For example, if the first method S100 correlates a motion type with a user activity but to a confidence score less than a threshold user activity confidence score, then the first method S100 can transmit the motion type (and start time, duration, and/or intensity, etc.) to the mobile computing device, and if the first method S100 correlates compressed motion data with a motion type but to a confidence score less than a threshold motion type confidence score, then the first method S100 can transmit the compressed motion data to the mobile computing device.

Block S110 of the first method S100 recites recording a set of raw motion data through a sensor incorporated into a wearable device. Generally, Block S110 functions to collect motion data through an accelerometer, a gyroscope, and/or other motion sensor incorporated into the wearable device, such as described in U.S. Provisional Application No. 61/710,867. Block S110 can record motion data continuously, or Block S110 can record motion data intermittently, such as when an acceleration and/or rotation of the wearable device exceeds a threshold acceleration and/or a threshold orientation change. Block S110 can also store raw motion data on non-volatile computer memory within the wearable device, such as in flash memory.

In one implementation, Block S110 collects acceleration data along three axes from a three-axis accelerometer within the wearable device and rotation data about (the) three axis from a three-axis gyroscope within the wearable device. Block S110 can also timestamp each set of six acceleration and rotation data points, such as with an absolute time (e.g., a GPS time, an approximated UTC time) or with a relative time (e.g., a local countdown or count-up time within the wearable device). Block S110 can collect data from the motion sensors at any suitable rate, such as at 100 Hz, 17 Hz, or 1.2 Hz, or at any other suitable rate. However, Block S110 can record raw motion data in any other way and according to any other trigger or event, and Block S110 can store raw motion data in any other suitable way or pass raw motion data directly to Block S120, as described below.

Block S120 of the first method S100 recites generating compressed motion data from the set of raw motion data. Generally, Block S120 of the first method S100 functions to compress raw motion data up to a first (e.g., lowest) compression level. In one implementation, Block S120 applies a lossless data compression technique or algorithm (e.g., a Lempel-Ziv compression method) to reduce a size (i.e., a number of bits) of the raw motion data by identifying and eliminating statistical redundancy within the raw motion data. In another implementation, Block S120 applies a lossy data compression technique or algorithm to reduce a size of the raw motion data by identifying unnecessary information and removing it. Block S120 can implement a source coding technique or algorithm to compress raw motion data before the raw data is stored locally on the wearable device—in other words, Block S120 can compress raw motion data in real time. Alternatively, Block S120 can implement data compression methods to reduce the size of raw motion data previously stored on the wearable device.

In one implementation, Block S120 converts raw motion data into quaternions. For example, for motion data with a particular timestamp, Block S120 can convert accelerations along three axes (i.e., output from a three-axis accelerometer within the wearable device) into one quaternion and rotations about three axes (i.e., output from a three-axis gyroscope with the wearable device) into a second quaternion. Block S120 can therefore generate quaternion acceleration and quaternion rotation (trajectory) pairs for raw motion data associated with each unique timestamp. Alternatively, Block S120 can convert accelerations along three axes and rotations about (the) three axes into a single quaternion defining a trajectory of the wearable device for each unique timestamp.

Block S120 can also implement machine learning techniques to improve data compression over time, such as to improve identification of important or key motion data within a set of motion data (over a period of time) to enable selective removal of less important motion from the set of motion data. However, Block S120 can implement any other data compression, data coding, and/or machine learning method, technique, or algorithm to convert raw motion data into compressed motion data.

Block S130 of the first method S100 recites, in a first mode, correlating the compressed motion data with a motion type. Generally, Block S130 functions to extrapolate a type of motion from a series of compressed motion data over time. In one implementation, Block S130 implements pattern recognition techniques to group compressed motion (i.e., accelerometer and/or gyroscope) data into classifications of recognized motion patterns. For example, Block S130 can implement a motion type algorithm to predict a walking motion, a running motion, a swinging motion (e.g., a tennis racket, a baseball bat), a drinking motion, a typing motion, etc. from a set of sequential quaternions output in Block S120.

Block S130 can therefore compress a set of compressed motion data over a period of time (e.g., two seconds, ten second, one minute) into a single motion classifier. For example, Block S130 can compress motion data recorded over a period of time into a single motion classifier including a motion type, a start time (e.g., based on an absolute time or a local time), an end time, a duration, and/or an intensity or speed. Block S130 can further identify repetitions of the same motions type and output a single motion classifier for multiple similar motions, such as a motion classifier that includes one motion type and a start time, duration, and intensity for each cycle of the motion type. Block S130 can therefore analyze accelerometer and/or gyroscope data recorded over time through sensors within the wearable device to classify how the user is moving within a period of time into a single motion type (and start time, duration, intensity, etc.).

Block S130 can select the motion type from a list of defined motion types. In one implementation, Block S130 accesses a list of motion types, wherein each motion type is associated with time-dependent acceleration and/or orientation characteristics and a textual descriptor (e.g., “swing,” “step,” or “hand-to-mouth”), a bit-type description (e.g., “01001” associated with “swing”, “0011” associated with “step”, “0101” associated with “hand-to-mouth”), and/or other type of descriptor. In this implementation, Block S130 can implement a motion type algorithm to match compressed motion data for one or more timestamps to a particular motion type. Alternatively, Block S130 can implement a non-parametric model (e.g., template matching) to match compressed motion data for one or more timestamps to a particular motion type. In this implementation, Block S130 can thus store the identified motion type in a string or array including the descriptor of the identified motion type, a start time, an end time, an intensity, and/or a number of sequential cycles, etc.

Block S130 can also implement unsupervised machine learning to improve classification algorithms for motion types. Block S130 can further interface with an input region on the wearable device and/or with the paired mobile computing device to verify identified motion types, and Block S130 can thus implement supervised or semi-supervised machine learning to improve motion type recognition faculties. However, Block S130 can function in any other way to extrapolate a type of motion from the compressed motion data and to store the identified motion type and associated metadata (i.e., start time, duration, etc.).

Block S140 of the first method S100 recites, in a second mode, transmitting the compressed motion data to a paired mobile computing device if the compressed motion data is not correlated with a motion type. Generally, Block S140 functions to handle transmission of compressed motion data (output in Block S110) to the mobile computing device if Block S130 fails to match the compressed motion data to a motion type or if Block S130 fails to match the compressed motion data to a motion type with a suitable degree of confidence. In one example, if Block S130 fails to identify a motion type with a confidence score greater than 90%, Block S140 can identify a sequence of compressed motion data over a period of time that appears to correspond to a single motion type and then transmit the sequence of compressed motion data to the mobile computing device, such over Bluetooth, Wi-Fi, or other wireless communication protocol. In another example, Block S140 can transmit, to the mobile computing device, all compressed motion data that Block S130 is unable to correlate with a motion type. In yet another example, if Block S130 correlates a first set of motion data with a first motion type that is well-known (e.g., a step) or associated with a motion recognition algorithm that is well-established (e.g., through machine learning over a relatively long period of time, such as months), Block S130 can pass the first motion type directly to Block S160, but if Block S130 correlates a second set of motion data with a second motion type that is new or not as well established (e.g., a handwriting motion, a shaving motion), both Block S160 can transmit the second motion type and Block S140 can transmit associated compressed motion data (or motion data that is further compressed beyond the compressed motion data) to the mobile computing device. In this example, the mobile computing device can locally check the second motion type against the compressed motion data to verify the second motion type and/or transmit the second motion type and the compressed motion data to a remote computer system for verification. Block S140 can therefore handle transmission of compressed motion data to the paired mobile computing device based on the identified motion type, a confidence score (or degree of confidence) in the correlated motion type, etc. However, Block S140 can transmit compressed motion data to the mobile computing device in any other way and according to any other trigger or event.

Block S150 of the first method S100 recites, in a third mode, correlating the motion type with a user activity. Generally, Block S150 functions to extrapolate a user action from a sequence of (the same or dissimilar) motion types (and associated meta data) identified in Block S130. For example, Block S130 can identify a sequence of step motions including meta data defining a duration. In this example, Block S130 can compare the number of steps in the sequence of step motions with the duration of the set of step motions to determine if the user is walking or running and an intensity of the user's motion (e.g., walking, jogging, running, sprinting, miles-per-hour, etc.). In another example, Block S130 can identify a sequence of hand-to-mouth motions with meta data defining a start time, a duration, and orientations of each hand-to-mouth motion. In this example, Block S150 correlate a portion of the hand-to-mouth motions with drinking and another portion of the hand-to-mouth motions with eating based on the orientation of each hand-to-mouth motion, compare the start time with a local approximation of UTC time, and thus determine if the user is eating breakfast, lunch, dinner, a snack, etc. and the size of the meal. In yet another example, Block S130 can identify a series of swinging motions—including meta data indicating increasing intensity—followed by a series of step motions, and Block S150 can correlate the swinging motions and steps with a hole of golf. In this example, Block S150 can further track the number of (series of) swing motions, the number of steps, and the duration of the event to determine how many holes a user played, an intensity of play throughout the round, estimate the user's score, etc. In another example, Block S130 can identify sedentary periods followed by an orientation change with meta data including start time and duration, and Block S150 can determine that the user is sleeping, identify a current user sleep cycle, and predict a wake time for the user, such as described in U.S. Provisional Application No. 61/710,869, filed on 8 Oct. 2012, which is incorporated herein in its entirety by this reference. Block S150 can therefore apply activity characterization algorithms, pattern recognition, machine learning techniques, etc. to correlate one or more motion types output in Block S130 with a user activity or action.

Block S150 can also tag the identified user activity with meta data including any one or more of start time, end time, duration, intensity, etc. of the user activity. However, Block S150 can function in any other way to correlate the motion type with a user activity.

Block S160 of the first method S100 recites, in a fourth mode, transmitting the motion type to the paired mobile computing device if the motion type is not correlated with a user activity. Generally, Block S160 functions to handle transmission of one or more motion types and associated meta data (output in Block S130) to the mobile computing device if Block S150 fails to match the motion type to a user activity or if Block S150 fails to match the motion type to a user activity with a suitable degree of confidence. In one example, if Block S150 fails to identify a user activity with a confidence score greater than 50%, Block S140 can select a sequence of identified motion types that appears to correspond to a single user activity and then transmit the sequence of motion types and associated meta data to the mobile computing device, such over Bluetooth, Wi-Fi, or other wireless communication protocol. In another example, Block S160 can cooperate with Block S140 to transmit, to the mobile computing device, a combination of identified motion types output in Block S130 and compressed motion data output in Block S120, such as motion types and compressed motion data that are disjoint sets in time or that overlap (i.e., intersect in time) based on confidence levels for identified motion types or motion type algorithms. In yet another example, if Block S150 correlates a first set of motion types with a first user activity that is well-known (e.g., walking, lifting weights, eating) or associated with an activity recognition algorithm that is well-established (e.g., through machine learning over a relatively long period of time, such as months), Block S150 can pass the first user activity directly to Block S170, but if Block S150 correlates a second set of motion types with a second user activity that is new or not as well established (e.g., shaving, petting a dog), both Block S160 can transmit the second set of motion types and Block S170 can transmit the second user activity to the mobile computing device. In this example, the mobile computing device can locally check the second user activity against the motion types and meta data to verify the second user activity and/or transmit the second user activity and the motion types and meta data to a remote computer system for verification. Block S160 can therefore handle transmission of motion types (and associated meta data) to the paired mobile computing device based on a correlated motion type, a confidence score (or degree of confidence) in the correlated user activity, etc. However, Block S160 can transmit identified motion types and (corresponding meta data) to the mobile computing device in any other way and according to any other trigger or event.

Block S170 of the first method S100 recites, in a fifth mode, transmitting the user activity to the paired mobile computing device. Generally, Block S170 functions to handle transmission of one or more identified user activities and associated meta data (output in Block S150) to the mobile computing device, such as if Block S150 matches the motion type to a user activity with a suitable degree of confidence. In one example, if Block S150 identifies a user activity with a confidence score greater than 95%, Block S110 can transmit the identified user activity and corresponding meta data, output in Block S150, to the mobile computing device, such over Bluetooth, Wi-Fi, or other wireless communication protocol. In another example, Block S110 can cooperate with Block S160 to transmit, to the mobile computing device, a combination of identified motion types output in Block S130 and identified user activities output in Block S150, as described above. However, Block S110 can transmit identified user activity data to the mobile computing device in any other way and according to any other trigger or event.

Block S140 can transmit compressed motion data to the mobile computing device at regular intervals while the wearable device and the mobile computing device are wireless connected, such as every ten minutes or every hour. Alternatively, Block S140 can transmit compressed motion data to the mobile computing device whenever the wearable device and the mobile computing device connect after a period without communication, such as whenever the wearable device and the mobile computing device wirelessly connect (e.g., ‘sync’) after four hours without communication. Similarly, Block S140 can transmit compressed motion data to the mobile computing device whenever the wearable device and the mobile computing device wirelessly connected within a specified time window, such as between 9 PM and 12 AM everyday. Block S160 and Block S110 can implement similar functionalities to transmit motion types and user activities to the mobile computing device. Blocks S140, S160, and S110 can also cooperate to prioritize data transmitted to the mobile computing device. For example, Block S110 can first transmit an identified user activity of a greatest confidence score and/or of a greatest duration since a previous sync with the mobile computing device, Block S160 can subsequently transmit a motion type of a greatest duration and/or number of repetitions since the previous sync with the mobile computing device, and Block S140 can subsequently transmit compressed motion data of a greatest duration since the previous sync with the mobile computing device. However, Blocks S140, S160, and S110 can prioritize transmission of compressed motion data, motion types, and user activities to the mobile computing device, respectively, in any other way any according to any other schema.

Generally, the first method S100 can dynamically select data compression levels (e.g., quaternions, motion types, user activities) for raw motion data output from motion sensors output within the wearable device by selectively implementing Block S120, the first mode in Block S130, and/or the third mode in Block S150. The first method S100 can further selectively transmit motion data in one or more compression levels by selectively implementing the second mode in Block S130, the fourth mode in Block S150, and/or the fifth mode in Block S110. As described above, the first method S100 can apply various compression levels to raw motion data output from one or more motion sensors within the wearable device based on confidence levels of compressed outputs, confidence in motion type or user activity models implemented by the first method S100 to derive a motion type or user activity from raw or compressed motion data, etc. The first method S100 can also implement machine learning and other techniques to improve compression, motion type, and user activity models or algorithms over time, such as supervised or semi-supervised machine learning through communication with the paired mobile computing device that verifies compressed motion data outputs from the wearable device. However, the first method S100 can function in any other way to classify motion data collected through motion sensors within a wearable device worn by a user.

### 2. Second Method

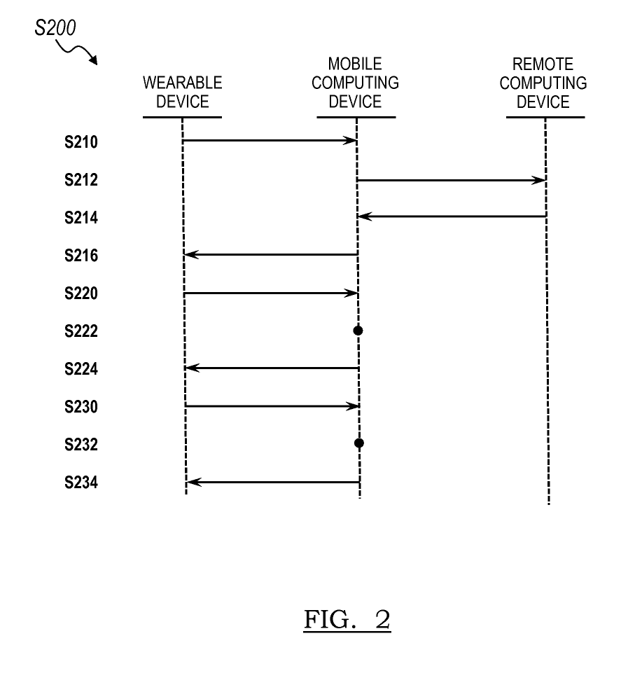

As shown in FIG. 2, a second method S200 for classifying a user action includes, in a first mode: receiving a compressed motion data from a wearable device in Block S210; transmitting the compressed motion data to a remote computer system if the compressed motion data cannot be correlated with a motion type within a defined confidence score Block S212; receiving a user activity from the computer system Block S214; and transmitting an updated motion type algorithm to the wearable device in response to receiving the user activity Block S216. The second method S200 also includes, in a second mode: receiving a motion type from the wearable device Block S220; correlating the motion type with a user activity Block S222; and transmitting an updated user activity algorithm to the wearable device in response to correlating the motion type with the user activity Block S224. The second method S200 further includes, in a third mode: receiving a user activity from the wearable device Block S230; confirming the user activity based on a location of the user Block S232; and transmitting an updated user activity algorithm to the wearable device in response to checking the user activity Block S234.

Generally, the second method S200 functions to interface with the first method S100 and wearable device described above to receive and handle compressed user motion data and to update the motion models and/or algorithms implemented by the first method S100 on the wearable device to improve local identification of motion types and user activities on the wearable device. The second method S200 can therefore be implemented as or within a native application executing on mobile electronic device carried by a user, such as a smartphone, a tablet, a smart watch, smart glasses, etc. For example, the second method S200 can be implemented within a native application that supports multiple internal wellness applications to support and/or improve user wellness, as described in U.S. patent application Ser. No. 14/048,956. The second method S200 can communicate with the wearable device via communication modules within the mobile computing device, such as over Bluetooth or Wi-Fi communication protocol. The second method S200 can also communicate with a remote computer network (e.g., a remote server, a remote database), such as through an Internet connection via Wi-Fi or cellular communication protocol.

One or more Blocks of the second method S200 can be implemented in real time, such as soon after compression motion, motion type, and/or user activity data is generated by and received from the wearable device. Alternatively, Blocks of the second method S200 can be implemented with a delay or latency, such as after a period of silence between the wearable device and the mobile computing device and once the wearable device syncs with the mobile computing device.

Block S210 of the second method S200 recites, in the first mode, receiving a compressed motion data from a wearable device. Generally, Block S210 functions to interface with Block S140 of the first method S100 to receive compressed motion data from the wearable device, wherein compressed motion data is smaller in size (i.e., fewer bits) than raw motion data collected by the motion sensor(s) within the wearable device but larger in size than motion type (and meta data) and user activity data transmitted from the wearable device in Blocks S160 an S170 and received in Blocks S220 and S230, respectively. For example, Block S210 can receive compressed motion data from the wearable device over Bluetooth or Wi-Fi communication protocol. However, Block S210 can function in any other way to receive compressed motion data from the wearable device.

Block S212 of the second method S200 recites, in the first mode, transmitting the compressed motion data to a remote computer system if the compressed motion data cannot be correlated with a motion type within a defined confidence score. Generally, Block S212 attempts to correlate the compressed motion data with a motion type as described above but transmits the compressed motion data (or a form thereof) to the remote computer system if a motion type cannot be ascertained or if a motion type cannot be identified within a suitable confidence score. Therefore, Block S212 can function to distribute data analysis to another computer system (e.g., ‘the cloud’) if Block S130 executing on the wearable device and the second method S200 executing on the mobile computing device cannot suitably identify a motion type from the compressed motion data. Block S212 can also push other relevant data, such as a GPS location of the mobile computing device (correlated with the location of the user), a user calendar event (e.g., stored on the mobile computing device or accessed from the Internet by the mobile computing device), meal details entered into the mobile computing device by the user, a user health goal or program (described in U.S. patent application Ser. No. 14/048,956), etc.—any of which can be associated with a time and matched to a compressed data including a timestamp—to the remote computer system. The remote computer system can thus implement such additional data to identify a motion type and/or a user activity from the compressed motion data.

Block S214 of the second method S200 recites, in the first mode, receiving a user activity from the computer system. Generally, Block S214 functions to communicate with the remote computer system to retrieve an identified motion type (or user activity) corresponding to compressed motion data sent to the remote computer system in Block S212. Additionally or alternatively, Block S214 can receive a motion type from the remote computer system and apply the received motion type to correlate the corresponding compressed motion data with a user activity.

Block S214 can also retrieve an updated motion type algorithm and/or an updated user activity algorithm from the remote computer system, such as a user activity algorithm implementable by the second method S200 on mobile computing device and/or a motion type algorithm implementable by the first method S100 executing on the mobile computing device. For example, the remote computer system can implement machine learning techniques to improve a motion type algorithm over time, and Block S214 can receive the updated algorithm from the remote computer system, and Block S216 can push the updated algorithm to the wearable device. Alternatively, Block S214 can implement machine learning techniques on the mobile computing device to generate the updated motion type algorithm and the user activity algorithm. Block S214 and/or the remote computer system can implement machine learning to update a motion type or user activity algorithm generally, that is, for substantially all users or for users of similar demographic, location, and/or gender, etc., or Block S214 and/or the remote computer system can implement machine learning to update a unique motion type or user activity algorithm specifically for a particular user associated with the particular mobile computing device and/or the particular wearable device.

Alternatively, Block S212 can implement methods and techniques described above in the first method S100 to correlate the compressed raw data with a motion type and/or an activity. Block S212 can also apply a GPS location of the mobile computing device, a user calendar event, meal details entered into the mobile computing device by the user, a health goal or health program enlisted by the user, etc. to further inform identification of a motion type or a user activity from the compressed raw data. For example, Block S212 can compare a location of the mobile computing device at a time defined by a timestamp tagged to the compressed motion data to verify with a suitable degree of confidence that minimal acceleration at the wearable device corresponds to sleeping and not to working at a desk for an extended period of time. Block S212 can thus apply the identified motion type and/or the identified user activity to teach a motion type algorithm or a user activity model, and Block S216 can push the algorithm and/or model to the wearable device, as described below.

Block S216 of the second method S200 recites, in the first mode, transmitting an updated motion type algorithm to the wearable device in response to receiving the user activity. Generally, Block S216 functions to push an updated (e.g., improved) motion type algorithm to the wearable device such that the first method S100 executing on the wearable device can implement the updated motion type algorithm (e.g., in Block S130) locally to correlate compressed motion data with a motion type. However, Block S210, S212, S214, and S216 can function in any other way in the discrete first mode to collect and handle compressed motion data received from the wearable device and to update the wearable device accordingly.

Block S220 of the second method S200 recites, in the second mode, receiving a motion type from the wearable device. Generally, Block S220 functions to interface with Block S160 of the first method S100 to receive a motion type (and corresponding meta data) from the wearable device. Block S220 can implement methods or techniques similar to those of Block S210 described above, such as by receiving a motion type from the wearable device over Bluetooth or Wi-Fi communication protocol.

Block S222 of the second method S200 recites, in the second mode, correlating the motion type with a user activity. Similarly to Block S212 described above, Block S220 can analyze one or more motion types (and associated meta data) in conjunction with mobile computing device data and user data stored on the mobile computing device, such as a GPS location, a calendar event, trends in user behavior, a time of day, meal details, selected health or wellness programs, etc. to further compress one or more motion types received from the wearable device into a user activity. For example, raw data over collected over a period of thirty minutes can be 1 MB in length, but compressed raw data (received in Block S210) for the same period of time can be 200 kB in length, motion types (received in Block S220) for the same period of time can be 50 kB in length, and a user activity identified in Block S222 can be 10 kB in length.

Like Block S212 and/or Block S214, Block S222 can generate an updated user activity model to convert a motion type to a user activity. Block S222 can generate the updated user activity model that is specific to the user, such as dependent on time-related daily habits, user preferences, or selected health goals. Alternatively, Block S222 can interface with the remote computer system to improve a generic user activity model applicable to all users, or to users of the same gender, to users of the same demographic, etc.

Block S224 of the second method S200 recites, in the second mode, transmitting an updated user activity algorithm to the wearable device in response to correlating the motion type with the user activity. Block S224 can thus implement techniques similar to Block S216 described above to update or sync an activity model or algorithm on the wearable device with the updated user activity model generated in Block S222.

Block S230 of the second method S200 recites, in the third mode, receiving a user activity from the wearable device. Generally, Block S230 functions to interface with Block S170 of the first method S100 to receive a user activity (and corresponding meta data) from the wearable device. Block S230 can implement methods or techniques similar to those of Block S210 and/or Block S220 described above.

Block S232 of the second method S200 recites, in the third mode, confirming the user activity based on a location of the user. Generally, Block S232 functions to verify a user activity identified in Block S150 of the first method S100 and received in Block S224 by comparing the user activity to additional user and/or mobile computing device data. For example, Block S232 can implement techniques or methods similar to those implemented in Block S212 and S222 described above, such as comparing a GPS location of the mobile computing device at a time defined by a timestamp tagged to the user activity to verify with a suitable degree of confidence the identified user activity matches typical activities, users trends, a user location, etc. at the associated time. In one example, Block S230 receives a user activity that indicates that the user played golf from 10 AM to 4 PM on a Saturday based on a series of swinging motions followed by a series of steps, but Block S232 determines that the mobile computing device (and therefore the user) was near tennis court but not a golf course, and Block S232 can thus modify the user activity (and/or request and re-analyze corresponding motion types from the wearable device) to indicate that the user was playing tennis—rather than playing golf—from 10 AM to 4 PM on the Saturday. However, Block S232 can compare a user activity received from the wearable device in Block S230 in any other suitable way.

Block S232 can further implement (supervised or semi-supervised) machine learning to update a user activity model based on the verified or modified user activity, and Block S234 can push the updated user activity model to the wearable device.

Block S234 of the second method S200 recites, in the third mode, transmitting an updated user activity algorithm to the wearable device in response to checking the user activity. Block S234 can thus implement techniques similar to Block S216 and Block S224 described above to update or sync an activity model or algorithm on the wearable device with the updated user activity model generated in Block S232.

The second method S200 can therefore stream compressed motion data, motion types (and corresponding meta data), and user activities (and corresponding meta data) from the wearable device and/or receive such motion data from the wearable device intermittently. The second method S200 can subsequently identify a motion type and/or a user activity locally on the mobile computing device and implement machine learning techniques to modify, improve, and/or tailor a motion type algorithm and/or a user activity algorithm for the user and update algorithm(s) on the wearable device accordingly. The second method S200 can additionally or alternatively transmit available motion data to a remote computer system that compresses the motion data further, and the second method S200 can receive a motion type and/or user activity from the remote computer system and again implement this data to improve a motion type and/or a user activity algorithm implemented by the second method S200 on the mobile computing device. The second method S200 can similarly implement this data to improve a motion type and/or a user activity algorithm implemented by the first method S100 on the wearable device.

Generally, the first method S100 and the second method S200 can function as an activity classification engine executing independently and together on a wearable device and a paired mobile computing device, respectively, to classify motion of a wearable device as a user activity. A behavior change engine executing on the mobile computing device can thus implement a user activity classified in the first method S100 and/or in the second method S200, such as described in U.S. patent application Ser. No. 14/048,956.

### 3. Third Method

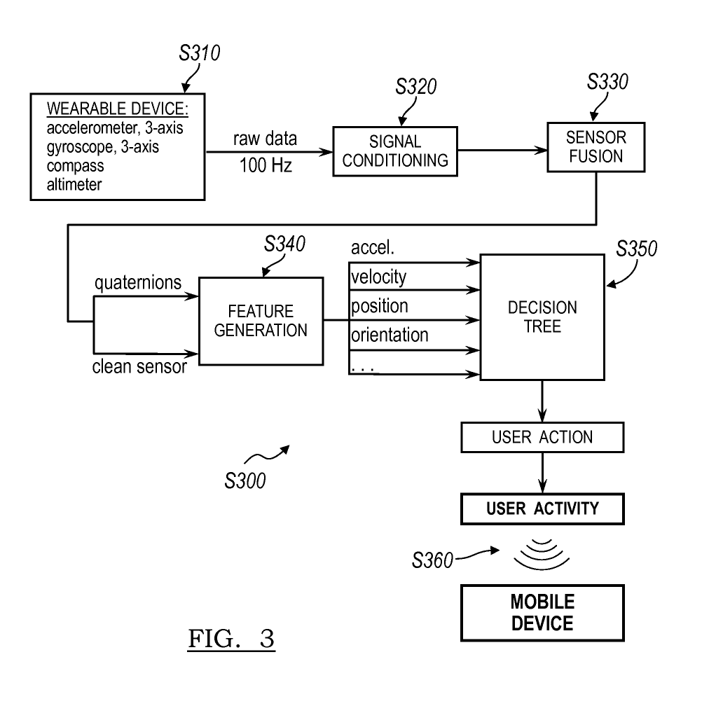

As shown in FIG. 3, a third method S300 for classifying motion of a user includes: during a second time interval, receiving a set of sensor data from a set of motion sensors arranged within a wearable device in Block S310; from the set of sensor data, generating a set of quaternions corresponding to instances within the second time interval in Block S330; generating a set of motion features from the set of quaternions in Block S340; transforming the set of motion features into a second action performed by the user within the second time interval in Block S350; and wirelessly transmitting a flag for the second action to an external computing device in response to a difference between the second action and a first action in Block S360, the first action determined from data received from the set of motion sensors during a first time interval immediately preceding the second time interval.

One variation of the third method S300 includes: during a second time interval, receiving a set of sensor data from a set of motion sensors arranged within a wearable device in Block S310; transforming the set of sensor data into a second action performed by the user within the second time interval in Block S350; calculating a confidence score for the second action in Block S350; and in response to a difference between the second action and a first action, wirelessly transmitting a flag for the second action, the confidence score for the second action, and a time tag corresponding to the second time interval to an external computing device in Block S360, the first action determined from data received from the set of motion sensors during a first time interval immediately preceding the second time interval.

Generally, the third method S300 can execute on a wearable device, as in the foregoing methods, to analyze and merge signals from various sensors within the wearable device, to identify a current action or activity performed by a user based on the signals, and to transmit an indicator (e.g., a flag) corresponding to the identified action or activity to an external device, such as if the identified action or activity corresponding to a current time interval differs substantially from a detected user action or activity corresponding to a time interval immediately prior to the current time interval. Like the first method S100 and the second method S200, the third method S300 can execute on a wearable device incorporating various sensors, such as a three-axis accelerometer, a three-axis gyroscope, a compass (or magnetometer), an altimeter, etc., as well as a battery and a wireless transmitter (or transceiver) that communicates with an external computing device (e.g., a smartphone, a tablet, or a laptop computer) as described above. In one example, the third method S300 merges and manipulates data collected through these various sensors onboard the wearable device to identify an action or activity of a user wearing the wearable device during a limited period of time (e.g., a one-second epoch), detects a difference in the current user action and a previous user action, and communicates an indicator for the new user action wirelessly to a paired computing device only when such difference between the current and previous actions is detected, thereby limiting wireless transmission of data from the wearable device to the external computing device to only instances in which the user's “state” changes and thus extending battery life of the wearable device.

Block S310 of the third method S300 recites, during a second time interval, receiving a set of sensor data from a set of motion sensors arranged within a wearable device. Generally, Block S310 functions like Block S110 of the first method S100 described above to collect raw data from various sensors within the wearable device. In one implementation, Block S310 samples the various sensors (e.g., an accelerometer, a gyroscope, and a magnetometer) within the wearable device at a constant rate (e.g., at 100 Hz) during the second (i.e., current) time interval. In an implementation in which the wearable device includes a three-axis accelerometer, a three-axis gyroscope, a magnetometer, and an altimeter, Block S310 can collect ten discrete values including three acceleration values, three rotation values, three magnetic readings (e.g., along X-, Y-, and Z-axes), and an altitude for each sample instance during the current time interval, such as one-hundred times per second during a two-second epoch defining the current time interval.

Block S310 can therefore sample one or more sensors within the wearable device at one or more instances during a time interval of a preset duration to generate data sets corresponding to the time, and subsequent Blocks of the third method S300 can then filter, fuse, calibrate, and/or analyze these data sets to determine an action or activity of the user during the time interval. Block S310 can further sample the sensor(s) at one or more instances during each succeeding time interval, and Blocks of the third method S300 can then manipulate data sets for each of these succeeding time intervals to identify an action or activity performed by the user during the corresponding time intervals.

Block S310 can transiently store raw (or filtered) sensor data pertaining to a particular time interval locally on the wearable device, such as in flash memory, and Block S310 can later erase these sensor data, such as upon generation of one or more features from the sensor data in Block S350 or upon determination of a user action from the sensor data in Block S360. Block S310 can therefore limit an amount of raw (or filtered) sensor data stored on the wearable device at any give time, such as by limiting stored sensor data to sensor data collected solely within a single time interval, to sensor data recorded over a duration of a single time interval (e.g., two seconds), or to sensor data collected within a limited number of time intervals (e.g., two time intervals) or a limited duration (e.g., four seconds). However, Block S310 can function in any other way and can execute at any other frequency to collect raw sensor data from one or more sensors arranged within the wearable device.

One variation of the third method S300 includes Block S320, which recites conditioning a set of raw sensor signals from the set of motion sensors. Generally, Block S320 functions to prepare (e.g., “clean up”) raw sensor data collected in Block S310 prior to manipulation of the sensor data in Blocks S330 and S340, such as by removing high-frequency noise from the sensor data. For example, Block S320 can apply a low-pass, high-pass, or bandpass filter to all or a subset of the raw sensor data received from corresponding sensors in the wearable device in Block S310. In this example, Block S320 can then output conditioned sensor data to Block S330 for subsequent fusion and quaternion generation. However, Block S320 can function in any other way to condition raw sensor data received from the various sensors.

Block S330 of the third method S300 recites, from the set of sensor signals, generating a set of quaternions corresponding to instances within the second time interval. Generally, Block S330 implements sensor fusion (and/or data fusion) techniques to remove noise, sensor drift, etc. from the (raw and/or or conditioned) sensor data and to merge sensor data into a set of quaternions. For example, Block S330 can implement methods and techniques similar to those of Block S120 of the first method S100 described above. In particular, Block S330 can select, from the sensor signals recorded during the time interval, a set of data points corresponding to a singular instance (or discrete period of time, e.g., ten milliseconds) within the time interval and then generate a single quaternion (or multiple quaternions) from the set of data points corresponding to the singular instance (or discrete period of time) within the time interval. Block S330 can then repeat this method to generate a signal quaternion (or multiple quaternions) for each other instance (or discrete periods of time) within the time interval to aggregate a set of quaternions corresponding to the current time interval. For example, Block S310 can sample motion sensors within the wearable device at a rate of 100 Hz over a current time interval, and Block S330 can generate a quaternion for each ten-millisecond interval during the current time interval. Therefore, in this example, for a one-second time interval, Block S330 can generate a set of 100 quaternions corresponding to the current time interval, including one quaternion per ten-millisecond interval during the one-second time interval.

Prior to generating a quaternion for a particular instance (or discrete period of time) within the time interval, Block S330 can “calibrate” outputs of one or more sensors within the wearable device. In one implementation, Block S330 applies gyroscope (i.e., rotation) data to accelerometer data to remove gravitational acceleration from acceleration data collected from a multi-axis accelerometer arranged within the wearable device in Block S310. For example, as the wearable device transitions from a static state—wherein the orientation of the wearable device is known by detecting gravity from the output of the accelerometer—to a motion state in which the wearable device translates and/or rotates, Block S330 can apply gyroscope data to acceleration data of the same instance (or discrete time period or time interval) to track rotation of the wearable device over time and the corresponding influence of gravity on outputs of the accelerometer. Block S330 can also apply altimeter data to accelerometer data to remove drift in the accelerometer data collected during the current (e.g., second) time interval. For example, Block S330 can track a change in vertical height (i.e., altitude) of the wearable device over the time interval (e.g., a two-second epoch) based on outputs of the altimeter, determine accelerations normal to the surface of the Earth during the time interval by removing gravity from the (raw or conditioned) acceleration data, twice-integrate these accelerations to estimate a vertical displacement during the time interval, and then correct the estimated vertical displacement from the accelerometer data based on a vertical displacement of the wearable device tracked through altimeter outputs.

Block S330 can additionally or alternatively implement compass data to correct drift in the output of the gyroscope arranged within the wearable device. For example, Block S330 can detect a change in orientation of the wearable device relative to a compass bearing across a time interval—less than or greater than the time interval in duration—based on outputs of the compass (or magnetometer) arranged within the wearable device, extract a rotation of the wearable device about an axis normal to the surface of the Earth during the time interval from gyroscope data, and correct drift in the gyroscope based on a comparison between the extracted rotation of the wearable device from gyroscope data and the detected change in orientation of the wearable device tracked through outputs of compass sensor. However, Block S330 can “fuse” data from various sensors within the wearable device in any other suitable way to reduce or correct sensor errors and/or to improve data collected from various sensors within the wearable device.

Block S330 can then convert these fused motion data into quaternions. In particular, Block S330 can generate a quaternion corresponding to a particular instance within the current time interval by fusing (raw or conditioned) sensor data throughout the time interval to remove sensor error (e.g., drift, gravitational effects, etc.) to create a set of corrected sensor signals and then combining data points corresponding to particular instances within the time interval into a corresponding quaternion. For example, for sensor data corresponding to a particular instance (or discrete period of time) within the current time interval, Block S330 can combine acceleration values for three perpendicular axes into one quaternion, combine rotation values about three perpendicular axes into a second quaternion, and combine an altitude value and a compass orientation (relative to the Earth) of the wearable device into a third quaternion for the particular instance within the time interval. Block S330 can then group these quaternions into a quaternion set for each instance (or discrete period of time) within the time interval, and Block S330 can then pass this quaternion set to Block S340 for manipulation into a set of features.

Alternatively, in another implementation, Block S330 can combine acceleration values along three axes, rotation values about three axes, altitude, and/or orientation (relative to the Earth), etc. of the wearable device into one quaternions specific to the instance within the time interval, and Block S330 can then pass this single quaternion to Block S340 for further manipulation. In this implementation, Block S330 can aggregate raw, conditioned, and/or fused sensor data corresponding to a particular instance within the time interval into a single quaternion according to a predefined quaternion generator rule, algorithm, or model. Block S330 can also implement multiple different quaternion generators (i.e., multiple different quaternion generator rules, algorithms, or models) to generate multiple discrete quaternions from the same sensor values corresponding to the same singular instance (or discrete period of time) within the time interval.

Block S330 can further “calibrate” the quaternion generator, such as within a discrete time interval or across multiple time intervals during operation of the wearable device. In one implementation, throughout operation of the wearable device (e.g., during repeated execution of the third method S300), Block S330 sets a calibration timer for a duration greater than the time interval and, in response to expiration of the timer, tests for a stable acceleration state of the wearable device. For example, for a time interval of one second, Block S330 can set a duration of five seconds for the calibration timer and, upon expiration of the calibration timer, test for a steady-state output of the accelerometer arranged within the wearable device (accounting for signal noise), such as may occur when the wearable device is not moving, when the wearable device is moving linearly at a substantially constant speed, or when the wearable device is rotating about a point at a relatively constant distance and at a relatively constant angular speed. In this implementation, Block S330 can then calibrate the quaternion generator, as described above, in response to detection of a stable acceleration state of the wearable device. In particular, Block S330 can recalibrate the quaternion generator for the known steady acceleration state of the wearable device when a steady acceleration test for the wearable device returns positive such that the quaternion generator compensates for drift in the output of one of more sensors within the wearable device. However, Block S330 can calibrate sensor output signals and/or the quaternion generator in any other suitable way, and Block S330 can function in any other way to fuse sensor data into “clean” (i.e., corrected) sensor data and to generate one or more quaternions accordingly.

Block S340 of the third method S300 generating a set of motion features from the set of quaternions. Generally, Block S340 functions to transform multiple quaternions corresponding to various instances (or discrete periods of time) within the current time interval into a set of features that characterize user motion within the current time interval. Block S340 can also merge (raw, conditioned, and/or fused) sensor signals with the set of quaternions corresponding to the current time interval to generate the set of motion features. For example, Block S340 can merge the set of sensor signals and quaternions corresponding to the current time interval into a first motion feature describing an acceleration of the wearable device during the current time interval relative to the Earth, a second motion feature describing a velocity of the wearable device during the current time interval relative to the Earth, and a third motion features describing an orientation of the wearable device during the current time interval relative to the Earth. As described below, Block S350 can then pass these features into a decision tree, algorithm, or other model to predict an action performed by the user during the time interval.

In one implementation, Block S310 samples sensors within the wearable device at a rate of 100 Hz, Block S330 outputs a quaternion for each sensor sample set (i.e., at a rate of 100 Hz), and Block S340 collects quaternions from Block S330 over a two-second time interval (i.e., 200 quaternions). Block S340 then combines the quaternions across the time interval into various features corresponding to the time interval. For example, Block S340 can generate an acceleration feature specifying a mean acceleration of the wearable device relative to the Earth during the time interval. Block S340 can also generate a velocity feature specifying a mean velocity of the wearable device relative to the Earth during the time interval, a position feature specifying a mean position of the wearable device relative to the Earth during the time interval, an orientation feature specifying a mean orientation of the wearable device relative to the Earth during the time interval, etc. Block S340 can group these features into a feature set corresponding to a particular time interval and then pass this feature set to Block S350 for correlation with a user action or activity during the time interval.

As shown in FIG. 3, Block S340 can implement a feature engine defining rules for generating features from quaternions and/or (raw, conditioned, and/or fused) sensor signals, such as rules for outputting mean values, weighted averages, standard deviations, or other composite values and/or statistics of quaternion and/or sensor data corresponding to the current time interval. Block S340 can also generate any number of features, such as one, two, or sixty features, by passing quaternion and/or sensor signal data into the feature engine.

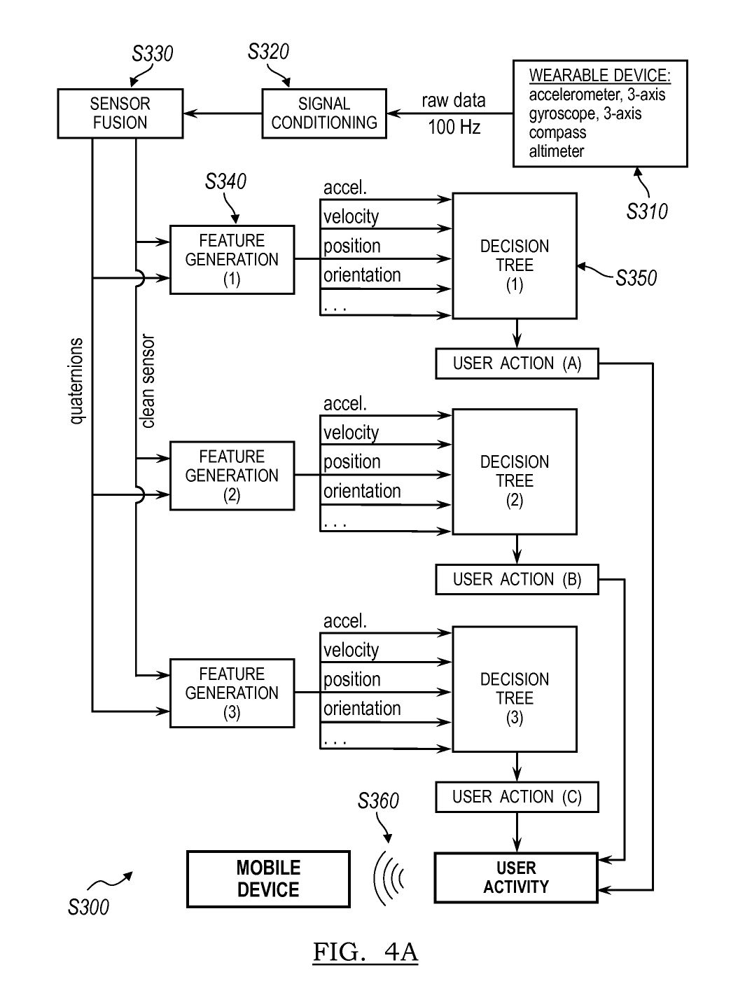

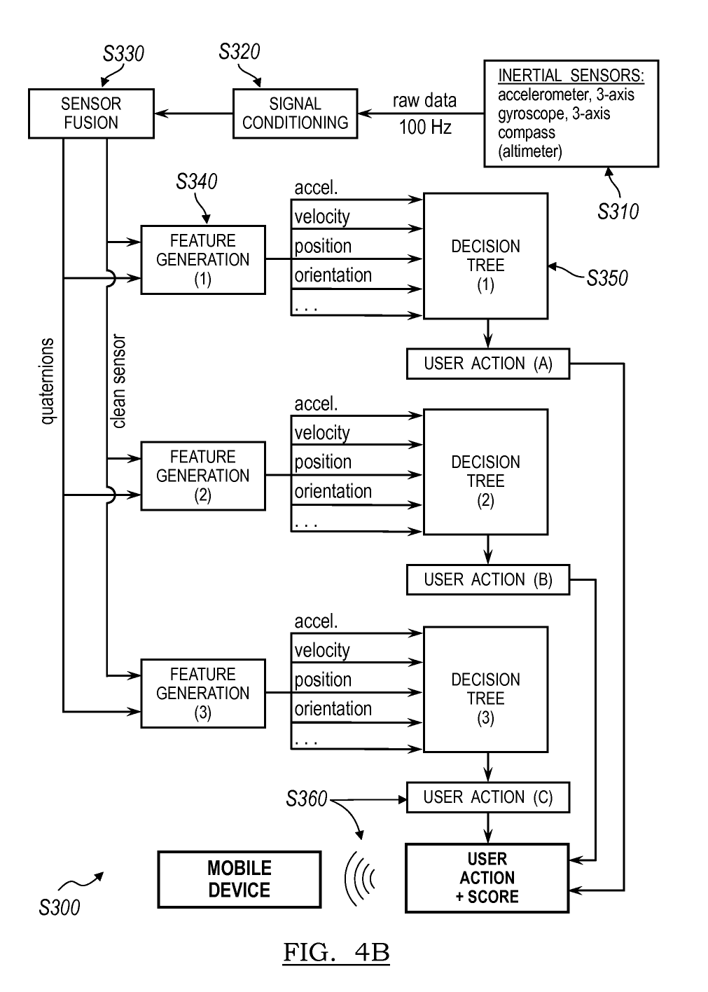

As shown in FIGS. 4A and 4B, Block S340 can also implement multiple discrete feature engines defining rules for generating unique sets of feature from the same set (or subset) of quaternions and/or sensor signals corresponding the time interval. Block S350 can then apply each (unique) feature set to a corresponding (unique) decision tree to output multiple discrete predictions for an action(s) performed by the user during the time interval (i.e., by generating one user action prediction per discrete feature set within the time period). In one example, Block S340 can implement a ranked set of feature engines, including a primary feature engine, a secondary feature engine, and a tertiary feature engine, etc., to generate a primary feature set, a secondary feature set, and a tertiary feature set, respectively, and Block S350 can pass the primary feature set into a primary model to generate a primary action prediction, pass the secondary feature set into a secondary model to generate a secondary action prediction, pass the tertiary feature set into a tertiary model to generate a tertiary action prediction, and confirm the primary action prediction with the secondary and tertiary action predictions and/or calculate confidence score for the primary action prediction based on a difference between the primary action prediction and the secondary and tertiary action predictions, as shown in FIG. 4A. Additionally or alternatively, Block S350 can combine multiple action predictions corresponding to the current time interval into a determined user activity characterized by a combination of actions within the time interval, as shown in FIG. 4B. However, Block S340 can function in any other way to generate a set of motion features from a set of quaternions corresponding to a time interval (i.e., time interval).

Block S350 of the third method S300 recites transforming the set of motion features into a second action performed by the user within the second time interval in Block S350. (Block S350 can similarly recite transforming the set of sensor signals into a second action performed by the user within the second time interval.) Generally, Block S350 functions to transform features generated in Block S340 into a predicted action (or activity) performed by the user during the corresponding time interval.

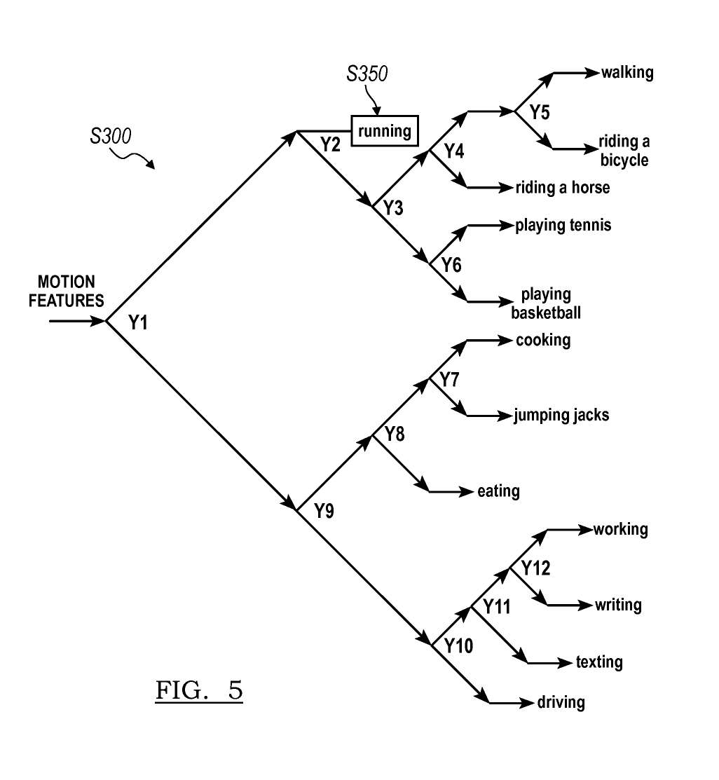

In one implementation, Block S350 passes features generated in Block S340 into a decision tree of n-dimensional hyperplanes with decision nodes defining equations and each end node defining a predicted user action, as shown in FIG. 5. In this implementation, each decision node can define an equation of the form)

yᵢ=A₀x₀+A₁x₁+A₂x₂+ . . . +Aₙxₙ,

wherein each of {A₀,A₁,A₂,+ . . . Aₙ} defines a coefficient and each of {x₁,x₂,+ . . . xₙ} includes a feature value output in Block S340. Block S350 can insert all or a subset of feature values—output in Block S340 and corresponding to the current time interval—into an equation at an initial decision node within the decision tree to calculate an output value y₁ for the initial decision node, move to a first subsequent decision node (or to a first subsequent end node) if the output value y₁ is less than a threshold value assigned to the initial decision node, and move to a second subsequent decision node (or to a second subsequent end node) if the output value y₁ exceeds the threshold value assigned to the initial decision node. Equations at each of the subsequent decision nodes can differ from the equation corresponding to the initial decision node, such as by differing in coefficient values and/or feature type variables, and Block S350 can repeat this process to calculate an output value y₂ for the selected subsequent decision node and can move through the decision tree accordingly until an end node is reached.

Each end node can be associated with one action, and Block S350 can thus pair the current time interval with an action associated with the final end node reached in the decision tree via the features values corresponding to the current time interval. Alternatively, each end node can be associated with two distinct actions, an equation, and a threshold value, and Block S350 can select from the distinct actions associated with the end node by passing feature values into the equation to generate an output value for the end node, selecting a first action associated with the node if the output value is less than the threshold value, selecting a second action associated with the node if the output value is greater than the threshold value, and pairing the current time interval with the selected action accordingly. For example, each end node of the decision tree can be associated with one (or more) of walking, running, riding a bicycle, riding a horse, playing tennis, playing basketball, doing pushups, doing jumping jacks, brushing teeth, cooking, drinking from a glass, drinking from a water fountain, driving a car, cooking, working at a computer, lounging, turning a page of a book, and unknown, etc., as shown in FIG. 5. Block S350 can thus implement a decision tree to transform a set of features corresponding to a single time interval (e.g., a two-second time interval including 200 sensor samples for each sensor/sensor axis within the wearable device) into a user action (or activity) during the time interval.

In the foregoing implementation, the equations, coefficients, and threshold values assigned to nodes within the decision tree can be preset or pre-programmed onto the wearable device. Alternatively, the third method S300 can implement machine learning or an other suitable technique to train or learn equations, coefficients, and/or threshold values assigned to nodes within the decisions tree. For example, a standard or “stock” decision tree can be uploaded and/or installed onto the wearable device, and the third method S300 can manipulate equations, coefficients, and/or threshold values defined within the stock decisions tree to improve action (and/or activity) classification for the particular user who wears the wearable device, thereby customizing the decision tree for the particular user, such as to accommodate for the user's height, weight, gait, primary activities, etc. Similarly, the third method S300—executing on the wearable device—can download new or updated equations, coefficients, and/or threshold values from a computing device (e.g., a smartphone). For example, a remote server can update decision tree values or generate whole new decision trees over time as a pool of participants wearing wearable devices increases and/or as greater volumes of user action data become available, the mobile computing device can retrieve these new decision tree values and/or decision trees from the remote server, and the wearable device can download the decision tree values and/or new decision trees, such as over Bluetooth or other wireless communication protocol, once a (wired or wireless) connection between the computing device and the wearable device is established. In this example, the third method S300 can receive—from the external computing device—a current (e.g., updated, customized, etc.) decision tree and then replace a previous decision tree stored on the wearable device with the current decision tree, and Block S350 can then implement the current decision tree to select an action prediction for the current time interval according to the corresponding set of quaternions and/or sensor data.

Block S350 can also select a particular decision tree from a set of available decision trees for selection of an action prediction for the current time interval. In one example, Block S350 accesses a database of decision trees, wherein each decision tree in the database is associated with one or more user characteristics (i.e., demographics), such as user age, gender, height, weight, build, mobility, medical condition, health status, etc., and Block S350 can retrieve demographic data of the user, such as from a user account or profile stored locally on the wearable device or from a native wellness application executing on an external computing device in communication with the wearable device. Block S350 can then filter the database of decision trees according to the user demographic data to select a decision tree that is particularly relevant to the user. Alternatively, Block S350 can select a particular decision tree from a set of available decision trees based on a time of day, a day of the week, a location of the wearable device (and therefore the user), the user's calendar or schedule, etc. For example, during a workday and/or when the wearable device is located within an office building, Block S350 can select a decision tree including end nodes associated with office-related tasks, such as typing, walking, meeting, presenting, etc. In another example, during portion of a day when the user is scheduled to be hiking, Block S350 can select a decision tree including end nodes associated with hiking-related tasks, such as walking, running, climbing, resting, cooking, eating, canoeing, rafting, etc. In this example, Block S350 can retrieve user calendar data from the external computing device and elect relevant decision trees for the user's schedule accordingly. However, Block S350 can elect a particular decision tree(s) from a set of available decision trees in any other way and according to any other variable or relevant factor. Yet alternatively, the external computing device and/or a remote computer network can implement any of the foregoing methods or techniques to select one (or more) relevant decision trees for the user, and Block S350 can retrieve and then implement this decision tree(s) accordingly. However, Block S350 can function in any other way to implement one or more decision trees specifically elected for the user to generate one or more action predictions from data collected during one or more time intervals.

As described above, Block S350 can also implement multiple decision trees, such as one unique decision tree (with corresponding unique equations, coefficients, threshold values, and/or actions) for each feature set output in Block S340 through one corresponding feature engine. Block S350 can thus generate multiple predictions of a user action during the current time interval and thus compare the predictions to verify an action and/or to assign a confidence score to a final predicted action for the current time interval. For example, if Block S340 implements three feature engines and Block S350 implements three corresponding decision trees, one of which outputs “running” and two of which output “riding a bicycle,” Block S350 can eliminate the “running” prediction, confirm the “riding a bicycle” prediction, and pass this latter action to Block S360.

In another example, Block S350 can apply a first combination of motion features in the set of motion features to a first function corresponding to a first node in the decision tree, select a second node of the decision tree according to an output value of the first function, apply a second combination of motion features in the set of motion features—differing from the first combination of motion features—to a second function corresponding to the second node, and generate an action prediction for the user for the current time interval according to an output value of the second function. In this example, Block S350 can further implement a second decision tree differing from the (first) decision tree to generate a selected action prediction for the current time interval, as described above, such as by applying a third combination of motion features in the set of motion features to a third function corresponding to a third node in a second decision tree, selecting a fourth node of the second decision tree according to an output value of the third function, applying a fourth combination of motion features in the set of motion features differing from the third combination to a fourth function corresponding to the fourth node, generating a second action prediction for the user during the time interval according to an output value of the fourth function. In this example, Block S350 can then confirm the (first) action prediction based on a comparison of the (first) action prediction with the second action prediction. In this example, the (first) decision tree can define a primary decision tree and the second decision tree can define a verification decision tree. Block S340 can implement a primary feature engine and a verification feature engine to output primary and secondary feature sets, and Block S350 can pass the primary feature set into the primary decision tree to select a “riding a bicycle” primary action prediction, can pass the secondary feature set into the verification decision tree to select a “running” verification action prediction, and can calculate a confidence score for the primary action prediction by comparing the primary action prediction with the verification action prediction. For example, Block S350 can calculate a confidence score of 70% for the “riding a bicycle” action prediction—rather than a confidence score of 100% if the primary and verification action predictions had matched—and Block S350 can then pass the “riding a bicycle” action prediction and the confidence score of 70% for the current time interval to Block S360. However, Block S350 can function in any other way to determine a current user action based on motion features corresponding to a time interval.

Block S350 can therefore further include calculating a confidence score for the action predicted for the current time interval. For example, Block S350 can generate a confidence score for the action predicted for the current time interval based on a difference between an action prediction generated from a first decision tree and a second action prediction generated from a second decision tree, as described above. Block S350 can assign numeric confidence scores to action predictions, such as a confidence scores between 50% and 100%, or Block S350 can assign, “low,” “medium,” and “high” confidence scores to action predictions.

Block S350 can additionally or alternatively group multiple action predictions for a single time interval, and Block S360 can transmit all or a portion of the group of multiple action predictions to the external computing device if the group of action predictions differs (by one or more action predictions) from an immediately-preceding group of action predictions generated at the wearable device for an immediately-preceding time interval. For example, Block S350 can implement three discrete decision trees to elect “walking,” “jogging,” and “riding a train” action predictions, and Block S360 can transmit this group of action predictions to the external computing device if relevant. Alternatively, Block S350 can implement one decision tree to elect multiple action predictions and can then pass this group of multiple action predictions to Block S360 accordingly.

In the foregoing implementation, Block S350 can alternatively combine multiple action predictions for the current time interval into a user activity for the time interval, such as shown in FIG. 4A. For example, Block S350 can elect a first action prediction for jogging, a second action prediction for spinning, and a third action prediction for swinging and then combine these action predictions into a predicted activity, such as “playing tennis,” for the single time interval. Alternatively, Block S350 can similarly combine action predictions across multiple time intervals into a single user activity for a period of time greater in duration than a single time interval. Block S360 can thus implement methods and techniques as described below to transmit new activity predictions (i.e., activity predictions that differ from immediately preceding activity predictions) to the external computing device, such as over wireless communication protocol. Block S350 can also calculate a confidence score for an activity prediction, and Block S360 can further transmit a confidence score with the corresponding activity prediction to the external computing device, such as according to a data standard, as described below.

In another implementation, Block S350 can pass features generated in Block S340 into an algorithm or other model to generate or elect an action prediction for the current time interval. For example, Block S350 can pass a subset of the motion features generated in Block S340 into a first algorithm to select a first prediction of the action, pass a subset of the motion features into a second algorithm to select a second prediction of the action, and identify the action for the current time interval based on the first prediction and the second prediction. However, Block S350 can implement any other one or more decision trees, algorithms, and/or models, etc. to transform features generated in Block S340 into one or more predictions for an action performed by the user during the current time interval.

Block S360 of the third method S300 recites wirelessly transmitting a flag for the second action to an external computing device in response to a difference between the second action and a first action, the first action determined from data received from the set of motion sensors during a first time interval immediately preceding the second time interval. Generally, Block S360 functions to transmit the action prediction for the current time interval—as elected in Block S350—to a paired computing device (e.g., a smartphone, a tablet) when the determined action for the current time interval differs from a determined action for a time interval immediately preceding the current time interval. For example, Block S350 can select a “running” action prediction for each two-second time interval during a time period starting at 10:15:48 and continuing through 10:37:56 followed by a “walking” action prediction for a two-second time interval between 10:37:56 and 10:37:58. In this example, Block S360 can transmit a single “running” flag to the computing device at soon after 10:15:50 following conclusion of the first two-second time interval between the period from 10:15:48 to 10:37:56 but withhold transmission of similar “running” flags for time intervals between 10:15:50 through 10:37:56. However, in response to the selected “walking” action prediction for the time interval from 10:37:56 to 10:37:58, which differs from the “running” action predictions for the previous 22:08 time period, Block S360 can transmit a “walking” flag to the computing device substantially soon after completion of the time interval at 10:37:58.

Block S360 can therefore compare each new action prediction selected in Block S350 to an immediately-preceding action prediction and transmit a flag for a current action prediction only when the current action prediction and a previous action prediction immediately preceding the current action prediction do not match. In particular, rather than transmitting a flag for every action prediction for every time interval to the external computing device, Block S360 can withhold transmission of data from the wearable device to the external computing device until a state of the wearable device (and therefore the user) changes, thereby reducing energy-intensive wireless data transmission from the wearable device and without substantially diminishing a quality, depth, or content of data shared within the external computing device. Block S360 can thus transmit only changes in detected actions of the user rather than every detected action at the wearable device, which can prolong battery life of the wearable device. For example, rather than transmitting a “walking” flag at the expiration of every two-second time interval while the user completes a twenty-minute walk (i.e., 600 transmissions of the same action), Block S360 can transmit a “walking” flag when the user starts walking and then a “static” flag when the user transitions from walking to standing in place. Thus, in this example, Block S360 can transmit only two action flags during the twenty-minute walk, including a single flag indicating that the user began walking and a second flag indicating that the user transitioned to an alternative action of standing still rather than, for example, transmitting 600 flags for the same “walking” action during the twenty-minute walking period. Because wireless data transmission can be energy intensive relative to collecting raw data and predicting a user action from the raw data as in Block S310, S320, S330, S340, and S350, Block S360 can thus substantially reduce power consumption by the wearable device (and/or other device similarly executing the third method S300) during classification and transmission of a user activity.

In one implementation, Block S360 includes, in response to a difference between the current (or “second”) action and an immediately-preceding (or “first”) action, wirelessly transmitting a flag for the current action, a confidence score for the current action, and a time tag corresponding to the current time interval. For example, Block S360 can interface with a local or world time from a clock executing on the wearable device to access a start time, center time, or end time for the current time interval and transmit this time with the action flag and the confidence score for the corresponding action prediction to the external computing device. Block S360 can also wirelessly transmit the flag, the confidence score, and the time tag for current time interval according to a data standard. For example, Block S360 can interface with a wireless transmitter or wireless transceiver arranged within the wearable device to transmit “7834237 (2014-06-20 10:09:32 am) (a=16, c=2),” wherein “7834237” is a unique identifier assigned to the corresponding time interval, wherein “2014-06-20 10:09:32 am” identifies the date and time of the time interval, wherein “a=16” indicates that the action “a” is “driving” according to a standard table correlating “16” to driving, “8” to running, “4” to walking, “2” to static, and “O” to unknown, and wherein “c=2” indicates that the confidence in the action tag is “high” according to a standard table correlating “2” to “high,” “1” to “medium,” “O” to “low.”

As described above, Block S360 can also transmit a group of action flags and a confidence score for each corresponding action prediction in the group, such as in response to addition of one or more new action predictions to the group for the current time interval relative to the action prediction group for the previous time interval, in response to elimination of one or more action predictions from the group for the current time interval relative to the action prediction group for the previous time interval, and/or in response to a confidence score for an action prediction for the group for the current time interval that differs from a confidence score for the action prediction in the action prediction group for the previous time interval, etc.

The third method S300 can then repeat for a subsequent (e.g., third) time interval succeeding the current (e.g., second) time interval. For example, Block S350 can store the flag for the second action in memory locally on the wearable device, and Block S310 can receive a second set of sensor signals from the set of motion sensors over the third time interval immediately succeeding the second time interval. Block S330 can then generate a second set of quaternions corresponding to instances within the third time interval from the second set of sensor signals, Block S340 can generate a second set of motion features from the second set of quaternions, and Block S350 can transform the second set of motion features into a third action performed by the user within the third time interval, and Block S360 can compare the third action to the second action. In particular, Block S360 can withhold wireless transmission of a flag for the third action to the external computing device in response to a (substantial) match between the second action prediction and the third action prediction, and Block S360 can wirelessly transmit the flag for the third action prediction to the external computing device in response to a difference between the second action prediction and the third action prediction, as described above.

In one variation, Block S360 recites transmitting a flag for a first action to an external computing device in response to a difference between the first action and the second action, the first action determined from data received from the set of motion sensors during a first time interval preceding the second time interval. Generally, in this variation, Block S360 functions to transmit a flag for a preceding action prediction when a new action is detected. For example, Block S350 can output a sequence of eighty-two “walking” action predictions corresponding to a sequence of eighty-two two-second time intervals and then a “running” action prediction for the eighty-third time interval. In this example, in response to the “running” action prediction, Block S360 can transmit a “walking” flag and a duration of the detected walking event, such as in the form of “2:44,” “164 seconds,” or “82 time intervals.” Subsequently, in this example, when Block S350 detects that the user has transitioned from running into an other action, Block S360 can transmit a flag for the running action and a corresponding duration of the running event. Block S360 can therefore calculate a duration of the first (i.e., immediately-preceding) action based on a sequence of contiguous time intervals associated with the first action. Therefore, in this variation, Block S360 can transmit a flag for an immediately-preceding action and a duration of a preceding action in response to detection of a different action in a current time interval. Block S360 can also transmit a start time (e.g., relative to local time, relative to a global time standard), an end time, etc. of the previous action in addition to the duration of the action and a flag for the action.

In the foregoing variations, Block S360 can alternatively store an action flag, a confidence score, an action duration, an action start time, and/or an action end time, etc. and transmit these data in response to receiving a request from an external computing device. For example, in response to selection of a new action prediction that differs from previous action prediction, Block S360 can attempt to wirelessly pair with an external computing device associated with the wearable device, and Block S360 can store data for the new (or the previous) action prediction locally on the wearable device if the wearable device fails to pair with the computing device. In this example, once the wearable device pairs with the computing device at a later time, Block S360 can retrieve the stored action prediction data and transmit these data to the computing device. Alternatively, Block S360 can wait for an action data request from the external computing device before transmitting the action data. For example, Block S360 can store action data only corresponding to new action predictions that differ from immediately-preceding action predictions, and Block S360 can transmit data for these limited number of action predictions to the external computing device in response to receiving a request for these data from the computing device.

Block S360 can further encrypt action prediction data prior to transmitting these data to the wearable device, such as according to Data Encryption Standard (DES), Triple Data Encryption Standard (3-DES), or Advanced Encryption Standard (AES).

However, Block S360 can function in any other way and in response to any other event to transmit a flag for a predicted action, a confidence score for the predicted action, and/or a time of the corresponding time interval to a mobile computing device.

The first method S100, the second method S200, and the third method S300 can similarly execute on any other suitable computing device. For example, the third method S300 can execute on a mobile computing device (e.g., a smartphone, a tablet) carried by a user to detect actions or activities performed by the user during over a time and to transmit detected actions or activities to an external computing device—such as a remote database or a remote computer network—when new action or activity predictions differ from immediately-preceding action or activity predictions. However, the first method S100, the second method S200, and the third method S300 can execute on any other suitable computing device to classify user actions or activities and to share user action or activity predictions with one or more external devices.

The systems and methods of the embodiment can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

We claim:

1. A method for classifying motion of a user, comprising:
during a second time interval, receiving a set of sensor signals from a set of motion sensors arranged within a wearable device;
from the set of sensor signals, generating a set of quaternions corresponding to instances within the second time interval;
generating a set of motion features from the set of quaternions;
transforming the set of motion features into a second action performed by the user within the second time interval; and
transmitting a flag for the second action to an external computing device in response to a difference between the second action and a first action, the first action determined from data received from the set of motion sensors during a first time interval immediately preceding the second time interval.

2. The method of claim 1, wherein receiving the set of sensor signals during the second time interval comprises sampling an accelerometer, a gyroscope, and a magnetometer at a constant sampling rate during the second time interval.

3. The method of claim 1, further comprising receiving a current action model from the external computing device and replacing a previous action model stored on the wearable device with the current action model, and wherein transforming the set of motion features into the second action comprises applying a subset of the set of motion features to the current model to determine the second action performed by the user within the second time interval.

4. The method of claim 3, wherein receiving the current action model from the external computing device comprises wirelessly downloading a set of functions corresponding to nodes of a decision tree, and wherein transforming the set of motion features into the second action comprises applying values defined in the set of motion features to functions corresponding to nodes in the decision tree to select the second action from a set of actions corresponding to end nodes in the decision tree.

5. The method of claim 1,
wherein generating the set of quaternions comprises, for each instance in a series of instances within the second time interval, generating a quaternion from a set of data points within the set of sensor signals corresponding to the instance;
wherein generating the set of motion features comprises merging sensor signals and quaternions corresponding to instances within the second time interval into the set of motion features; and
wherein transforming the set of motion features into the second action comprises passing a subset of the set of motion features into a decision tree.

6. The method of claim 5, further comprising selecting the decision tree from a set of available decision trees based on a demographic of the user.

7. The method of claim 5, wherein generating the set of motion features comprises merging the set of sensor signals and quaternions into a first motion feature describing an acceleration of the wearable device during the second time interval relative to the Earth, a second motion feature describing a velocity of the wearable device during the second time interval relative to the Earth, and a third motion features describing an orientation of the wearable device during the second time interval relative to the Earth.

8. The method of claim 5, wherein passing the subset of the set of motion features into the decision tree comprises applying a first combination of motion features in the set of motion features to a first function corresponding to a first node in the decision tree, selecting a second node of the decision tree according to an output value of the first function, applying a second combination of motion features in the set of motion features to a second function corresponding to the second node, the second combination of motion features differing from the first combination of motion features, and determining the second action according to an output value of the second function.

9. The method of claim 8, further comprising applying a third combination of motion features in the set of motion features to a third function corresponding to a third node in a second decision tree, selecting a fourth node of the second decision tree according to an output value of the third function, applying a fourth combination of motion features in the set of motion features differing from the third combination to a fourth function corresponding to the fourth node, determining the third action of the user during the second time interval according to an output value of the fourth function, and confirming the second action based on a comparison of the second action and the third action.

10. The method of claim 9, wherein confirming the second action comprises generating a confidence score for the second action based on a difference between the second action and the third action, and wherein transmitting the flag for the second action comprises wirelessly transmitting the flag for the second action and the confidence score for the second action to the external computing device further in response to the confidence score exceeding a threshold confidence score.

11. The method of claim 1, further comprising calculating a confidence score for the second action, wherein transmitting the flag for the second action comprises transmitting the confidence score, the flag for the second action, and a timestamp corresponding to the second time interval according to a data standard in response to detection of the second action that differs from the first action.

12. The method of claim 11, wherein transforming the set of motion features into the second action comprises passing a subset of the motion features into a first algorithm to select a first prediction of the action, passing a subset of the motion features into a second algorithm to select a second prediction of the action, and identifying the second action based on the first prediction and the second prediction, and wherein calculating the confidence score comprises calculating the confidence score based on a correlation between the first prediction and the second prediction.

13. The method of claim 1, further comprising
storing the flag for the second action in memory locally on the wearable device;
during a third time interval immediately succeeding the second time interval, receiving a second set of sensor signals from the set of motion sensors;
from the second set of sensor signals, generating a second set of quaternions corresponding to instances within the third time interval;
generating a second set of motion features from the second set of quaternions;
transforming the second set of motion features into a third action performed by the user within the third time interval;
comparing the third action to the second action; and
withholding transmission of a flag for the third action to the external computing device in response to a match between the second action and the third action.

14. The method of claim 1, further comprising conditioning the set of sensor signals, wherein generating the set of quaternions comprises generating the set of quaternions from conditioned sensor signals.

15. The method of claim 1, further comprising
setting a timer for a duration greater than the second time interval;
in response to expiration of the timer, testing for a stable acceleration state of the wearable device; and
in response to detection of a stable acceleration state of the wearable device, calibrating a quaternion generator generating quaternions from sensor signals received from the set of motion sensors.

16. The method of claim 1, further comprising generating a second set of motion features from the set of quaternions and transforming the second set of motion features into a third action performed by the user within the second time interval, wherein transmitting the flag for the second action comprises wirelessly transmitting the flag for the second action batched with a flag for the third action in response to a difference between a second action set and a first action set, the first action set defining the first action and corresponding to the first time interval, the second action set defining the second action and the third action and corresponding to the second time interval.

17. A method for classifying motion of a user, comprising:
during a second time interval, receiving a set of sensor signals from a set of motion sensors arranged within a computing device;
transforming the set of sensor signals into a second action performed by the user within the second time interval;
calculating a confidence score for the second action; and
in response to a difference between the second action and a first action, transmitting a flag for the second action, the confidence score for the second action, and a time tag corresponding to the second time interval to an external computing device, the first action determined from data received from the set of motion sensors during a first time interval immediately preceding the second time interval.

18. The method of claim 17, wherein transmitting the flag, the confidence score, and the time tag to an external computing device comprises wirelessly transmitting the flag, the confidence score, and a time tag for a local current time corresponding to a start of the second time interval according to a data standard in response to detection of the second action that differs from the first action.

19. The method of claim 17, wherein transforming the set of sensor signals into the second action comprises
for each instance in a series of instances within the second time interval, generating a quaternion from a set of data points within the set of sensor signals corresponding to the instance;
merging sensor signals and quaternions corresponding to instances within the second time interval into the set of motion features; and
wherein transforming the set of motion features into the second action comprises selecting a particular end node in a decision tree based on a subset of the set of motion features, the particular end node describing the second action.

20. The method of claim 17, wherein receiving the set of sensor signals from the set of motion sensors comprises recording signals from a gyroscope and an accelerometer arranged within the wearable device during the second time interval at least one second in duration.

21. A method for classifying motion of a user, comprising:
during a second time interval, receiving a set of sensor signals from a set of motion sensors arranged within a wearable device;
from the set of sensor signals, generating a set of quaternions corresponding to instances within the second time interval;
generating a set of motion features from the set of quaternions;
transforming the set of motion features into a second action performed by the user within the second time interval; and
transmitting a flag for a first action to an external computing device in response to a difference between the first action and the second action, the first action determined from data received from the set of motion sensors during a first time interval preceding the second time interval.

22. The method of claim 21, wherein transmitting the flag for the first action comprises calculating a duration of the first action based on a sequence of contiguous time intervals associated with the first action and wirelessly transmitting the flag for the first action and the duration of the first action.
